(19) United States
(12) Patent Application Publication
(10) Pub. No.: US 2004/0122701 A1
(43) Pub. Date: Jun. 24, 2004

(54) Systems and methods for integrating disease management into a physician workflow

(76) Inventors: Michael D. Dahlin, Austin, TX (US); Randolph B. Lipscher, Austin, TX (US); Charles Andrew Bergman, Lutherville-Timonium, MD (US); Eric Wohl, Austin, TX (US)

Correspondence Address: TOLER & LARSON & ABEL L.L.P., 5000 PLAZA ON THE LAKE STE 265, AUSTIN, TX, 78746, US

(21) Appl. No.: 09/992,035

(22) Filed: Nov. 23, 2001

Related U.S. Application Data
(60) Provisional application No. 60/252,820, filed on Nov. 22, 2000.

(51) Int. Cl. G06F 17/60; A61B 5/00

(52) U.S. Cl. 705/2; 600/300

(57) ABSTRACT

The present invention is directed to an electronic system and a method for integrating disease management information into a physician workflow. A method for presenting treatment algorithms to at least one user, the method may comprise: receiving or storing treatment algorithms from at least two disease management advisors; selecting a subset of treatment algorithms; and presenting the subset of treatment algorithms to the at least one user. For specifying medical diagnosis and treatment algorithms that may be integrated into a healthcare workflow of the electronic system, the electronic system may include: (a) a coordination server having one or more rules for selecting at least one treatment algorithm based on medical and demographic information about a patient; and (b) an interface for providing a plurality of questions related to one or more medical findings, the questions may be asked of the patient or entered about the patient and potential orders that may be executed for the patient. The electronic system may further enable access to a plurality of entities; each entity may specify one or more treatment algorithms to be included in one or more healthcare workflows. For example, one or more third party disease management advisors (DMAs) may be integrated into a physician workflow. An electronic medical record (EMR) device such as a wireless tablet which is generally used by healthcare professionals (HCPs) is leveraged to integrate a plurality of disease management algorithms with the traditional EMR workflow of an electronic medical record (EMR) system typically utilized for dispensing a variety of healthcare services. In particular, such integration provides a disease management EMR system for effective communication between a plurality of disease management advisors, a plurality of healthcare professionals, and a plurality of patients.

This application claims priority of U.S. patent application Ser. No. 09/______, filed Nov. 22, 2000 entitled: “SYSTEMS AND METHODS FOR INTEGRATING DISEASE MANAGEMENT INTO A PHYSICIAN WORKFLOW”, and is incorporated herein by reference in its entirety.

### TECHNICAL FIELD OF THE INVENTION

The present invention relates to the field of healthcare delivery and, in particular, to an electronic system that integrates disease management into a physician workflow and that can serve as a single point of integration for third party Disease Management Advisors (DMAs).

### BACKGROUND OF THE INVENTION

To improve efficiency of medical practitioners and to curtail the rising cost of providing health care, many attempts have been made to use computers or electronic medical records (EMR) systems to facilitate the delivery of a variety of healthcare services. Such systems have generally failed to efficiently integrate disease management with the physician or medical practitioner's workflow.

Typically, disease management uses specific diagnosis and treatment algorithms for different patients, diseases, and payers to improve patient outcomes, reduce side effects and/or complications, and/or reduce costs. It represents an aspect of data-driven medicine that provides healthcare professionals/practitioners/providers detailed instructions for specific situations based on quantitative studies rather than following a smaller number of more general rules based on more general studies or experience.

The number of disease management rules or algorithms that a healthcare practitioner will be expected to utilize will increase in the future as more quantitative data become available, as more treatment options or tests become available, as the range of costs of different treatment options widens, and as increasing numbers of payers or disease management advisors become involved in cost-control and quality-improvement of health care.

These factors pose significant challenges to healthcare practitioners because they must remember and apply large numbers of rules both because the set of rules for each disease increases as technology advances and because business concerns may further increase the set of treatment rules a physician must follow. Different patients with the same condition may be subject to different disease management algorithms if they have different disease management advisors.

Integrating these data-driven disease management algorithms into medical practitioner's workflows involves several challenges. First, it is a challenge to provide an effective communication from a Disease Management Advisor (DMA) to a Healthcare Practitioner (HCP). Second, communication of information/data from a HCP to a DMA can be difficult. Third, a real-time communication between a patient and a DMA could be difficult as well.

For communication from a DMA to a HCP, DMAs may transmit treatment algorithms to HCPs. However, it may be in a way that makes it unlikely the HCP will follow the algorithm. Many typical systems fail to integrate information provided by the algorithm into a physician's workflow to minimize disruption to normal patterns of practice while making the information available at the appropriate time. For example, telephoning a patient's disease management advisor while working with a patient would be extremely disruptive to a physician's practice and would inhibit widespread adoption of such algorithms. Current techniques are likely to be ineffective. For example, delivering batches of paper copies of disease management algorithms to practitioners would likely be ineffective because the copies would not be readily available when the practitioner is working with a patient. For example, posting a list of algorithms on the web—in HTML or XML form, for example—would likely be ineffective because it would require the practitioner to stop his or her normal workflow, search for the desired information, and then resume the workflow.

These treatment algorithms may be generic (e.g., a generic decision-flow diagram for diagnosing and treating respiratory distress in 50-60 year old males), context-sensitive suggestions integrated into a physician's workflow (e.g., “based on the generic decision flow algorithm and the findings present in the patient record, the next step in treatment is X”), or specific suggestions for diagnosing or treating a specific patient (e.g., a DMA may provide instructions to an HCP to perform a specific test on a specific patient.) These different cases may require different integration techniques.

Disease management treatment algorithms include medication step therapy, diagnosis algorithms, treatment algorithms, decision criteria for ordering certain tests, and the like. Disease management advisors may include specialized disease management advising companies, pharmacies, insurance companies, HMOs, government agencies, medical professional societies, medical specialists, experts in particular fields of medicine, pharmacy benefits management companies, advertisers, pharmacies, labs, academic organizations, medical information publishers, research institutions, organizers of medical studies, organizers of medication trials, and the like.

Handling multiple algorithms from many different DMAs poses a significant challenge that is further compounded by the multiple algorithms sent to HCP's in different situations. Different patients or conditions may have different algorithms for technical/medical reasons. Furthermore, different patients may have different advisors (and thus different algorithms) depending on their payer or payers (insurance company, health maintenance organization, benefits company, long-term disability insurer, life insurer, drug benefits company, etc.) The presence of many DMAs poses another challenge of providing treatment algorithms in different formats or at different locations.

Further, the DMAs communication of suggestions to HCPs should not interfere with the HCP's normal flow of work. For example, displaying a dialog box with additional DMA-sponsored questions at the end of a task may be unacceptable in many contexts.

As described, communication of information/data to a disease management advisor is another challenge faced by developers both from the DMA and to the DMA from the HCP. For example, after completion of a patient examination in a clinic, many typical systems lack a means for the HCP to send the DMA the results of that examination. Moreover, if a DMA sends a patient to an HCP for a laboratory test or physical examination, many typical systems lack a means to transmit the results of that test or examination back to the DMA.

Unfortunately, many typical techniques are ineffective at providing communication of information/data to disease management advisors. Manually sending the information (in electronic, paper, fax, or voice form) is cumbersome. Providing automatic access to the patients' medical records by the DMA raises privacy concerns (because it does not restrict what data may be accessed), and is cumbersome (because much of the information provided to the DMA is not of use to the DMA).

Communication between a patient and a disease management advisor is a further challenge. A DMA may occasionally or periodically ask a patient for information. For example, the DMA may ask the patient if they have taken their medication today. Further, the DMA may send instructions to the patient. For example, the DMA may ask the patient to go to a HCP for a test such as a blood glucose test.

Unfortunately, many typical techniques for providing this communication are cumbersome (e.g., phone calls, e-mail, paper, or pagers). Some researchers have proposed providing electronic access by external parties to patient pillboxes. However, in general, it is difficult to provide such electronic access, and can be even more difficult for disease management purposes.

### SUMMARY OF THE INVENTION

A system for specifying medical diagnosis and treatment algorithms that may be integrated into a healthcare workflow, the system may include: (a) a coordination server having one or more rules for selecting at least one treatment algorithm based on medical and demographic information about a patient; and (b) an interface for providing a plurality of questions related to one or more medical findings, the questions may be asked of the patient or entered about the patient and potential orders that may be executed for the patient. The system may further enable access to a plurality of entities; each entity may specify one or more treatment algorithms to be included in one or more healthcare workflows.

A system for specifying medical diagnosis and treatment algorithms that may be integrated into a healthcare workflow, the system may include: (a) one or more coordination servers having one or more rules for selecting at least one treatment algorithm based on medical and demographic information about a patient; (b) an interface for providing a plurality of questions related to one or more medical findings, the questions may be asked of the patient or entered about the patient and potential orders that may be executed for the patient; and (c) a distribution server that distributes information from the interfaces to the one or more coordination servers, receives one or more treatment algorithms from the one or more coordination servers, and transmits these one or more treatment algorithms to the one or more interfaces to be included in one or more healthcare workflows.

One embodiment of the invention is based on a method for presenting disease management algorithms to at least one user, the method may comprise: receiving or storing disease management algorithms from at least two disease management advisors; selecting a subset of algorithms; and presenting the subset of algorithms to the at least one user. In the method step of selecting, the at least one user may search with one or more selection criteria. Likewise, in the method step of presenting the subset of algorithms may be presented using a common display format. The method may further comprise an electronic healthcare workflow in which the step of selecting selects algorithms associated with medical or demographic information in an active patient's medical record. Alternatively, the method may further comprise an electronic healthcare workflow in which the step of selecting selects algorithms associated with one or more of the active patient's disease management advisors. In addition, the method may further comprise an electronic healthcare workflow in which the step of presenting comprises augmenting, modifying, or replacing the electronic healthcare workflow display with disease management algorithm information.

Another alternate embodiment of the invention is based on a computer-implemented method for integrating the display of disease management algorithms with an electronic medical record, the method comprising: providing an electronic healthcare workflow having one or more workflow tasks; providing one or more disease management algorithms; selecting one or more of the disease management algorithms; and presenting information or options specified by the disease management algorithms during the execution of one or more workflow tasks. The method step of selecting may further comprise utilizing relevant disease management algorithms based on the current patient's demographic information or medical information or both. The method step of presenting may further comprise augmenting, modifying, or replacing the options available or the data displayed at one or more workflow tasks in the electronic healthcare workflow. The displayed data may include a subset of algorithm elements that are displayed with the one or more workflow tasks or those algorithm elements that are associated with the specific workflow task. The method step of presenting may further comprise using a plurality of actions. The actions may include, but are not limited to, displaying a banner, augmenting a data-input template, modifying a data-input template, replacing a data-input template, displaying an icon, displaying a warning, requiring acknowledgement of a warning, sending an alert message to the healthcare practitioner, providing an input device that may be activated to execute specified orders, providing an auditory notification, providing a tactile notification, changing the workflow order, and/or introducing one or more steps to the electronic healthcare workflow order.

Another embodiment of the invention provides a healthcare EMR system using an integrated workflow routine, a disease management engine, a plurality of disease management algorithms, and an interface to provide communication between one or more external disease management advisors and one or more healthcare providers/professionals/practitioners for servicing one or more patients. The communication may be provided through the interface in conjunction with the integrated workflow routine, which may utilize one or more workflow mechanisms.

For example, in a workflow mechanism, a disease management advisor may create an appointment within the healthcare EMR system for a patient to schedule a visit with a healthcare professional at a clinic for a healthcare professional-patient encounter. The disease management advisor may transmit relevant information about the patient to the healthcare EMR system that is displayed by the integrated workflow routine during the patient's visit to the clinic. The disease management advisor may transmit a disease management algorithm to the disease management engine for augmenting the integrated workflow routine for the healthcare EMR system. Further, the disease management engine may transmit refill requests for the patient to the integrated workflow routine. In addition, the disease management engine, responsive to the disease management algorithm, may send a message to the healthcare professional containing information or suggestions for improving his/her practice of medicine. For example, during a specific task from the disease management algorithm, the disease management engine may send a message to the healthcare professional containing information or suggestions for improving his/her decision for the specific task. The disease management advisor may send the healthcare professional a directive to send specific information about the patient to the disease management advisor upon completion of the healthcare professional-patient encounter. In response to the directive, the healthcare professional may send information about the patient to the disease management advisor. The integrated workflow routine may send information about the patient from the healthcare EMR system to the disease management engine. For example, the information may include decisions or information entered into the integrated workflow routine during or for one or more past healthcare professional-patient encounters. Further, the disease management engine may analyze these decisions to evaluate their effectiveness, to generate suggestions for improvement, or to provide data generated to a medical data gathering device controlled by the integrated workflow routine which sends data to the disease management advisor.

Another alternate embodiment of the invention is based on a healthcare EMR system. The healthcare EMR system may include an integrated workflow routine, a disease management engine, a plurality of disease management algorithms, and an interface to provide communication between one or more external disease management advisors, one or more healthcare providers/professionals/practitioners, and one or more patients. The communication may be provided through the interface in conjunction with the integrated workflow routine, which may utilize one or more workflow mechanisms.

For example, in a workflow mechanism, a disease management advisor may direct a patient to make an appointment through the healthcare EMR system. Alternatively, a disease management advisor may create an appointment within the healthcare EMR system for a patient to schedule a visit with a healthcare professional at a clinic for a healthcare professional-patient encounter. The disease management advisor may transmit relevant information about the patient to the healthcare EMR system that is displayed by the integrated workflow routine during the patient's visit to the clinic. The disease management advisor may transmit a disease management algorithm to the disease management engine for augmenting the integrated workflow routine for the healthcare EMR system. The disease management engine may transmit refill requests for the patient to the integrated workflow routine. The disease management engine, responsive to the disease management algorithm, may send a message to the healthcare professional containing information or suggestions for improving his/her practice of medicine. For example, during a specific task from the disease management algorithm, the disease management engine may send a message to the healthcare professional containing information or suggestions for improving his/her decision for the specific task. The disease management advisor may send the healthcare professional a directive to send specific information about the patient to the disease management advisor upon completion of the healthcare professional-patient encounter. In response to the directive, the healthcare professional may send information about the patient to the disease management advisor. Further, the integrated workflow routine may send information about the patient from the healthcare EMR system to the disease management engine. For example, the information may include decisions or information entered into the integrated workflow routine during or for one or more past healthcare professional-patient encounters, and the disease management engine may analyze these decisions to evaluate their effectiveness. To generate suggestions for improvement, the disease management engine may query the patient to enter treatment compliance information and the health care system may transmit the results to the disease management engine and/or to the healthcare professional for integration with the integrated workflow routine. In response, the disease management engine may transmit instructions to the patient, to the healthcare professional, and/or to provide data generated to a medical data-gathering device controlled by the integrated workflow routine which sends data to the disease management advisor.

Another embodiment of the invention is based on an electronic media, comprising a program for performing the methods of the invention. Another embodiment of the invention is based on a computer program, comprising computer or machine-readable program elements translatable for implementing the methods of the invention.

The foregoing has outlined, rather broadly, the aspects of the present invention in order that the detailed description of the invention that follows may be better understood. Additional aspects of the invention that form the subject of the claims of the invention will be described hereinafter. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present invention and the advantages thereof, reference is now made to the following descriptions taken in conjunction with the accompanying drawings, in which:

FIG. 1 is a flow chart depicting a prior art EMR workflow of traditional electronic medical records (EM s) systems.

FIG. 2 is a flow chart showing an exemplary integration of disease management advisor (DMA) information and treatment algorithms into a physician/healthcare professional (HCP) workflow to provide an extended EMR workflow according to one embodiment of the present invention.

FIG. 3 illustrates an exemplary graphical user interface that may be used in an embodiment of the present invention for integrating DMA information from a plurality of disease management advisors (DMAs) and presenting that information to one or more healthcare professionals (HCPs).

FIG. 4 shows a display of an exemplary treatment algorithm for acute coronary syndrome consistent with one embodiment of the present invention.

FIG. 5 depicts an exemplary graphical user interface having an exemplary strategy integrated into a physician workflow for a healthcare professional (HCP) to write an electronic prescription for a medication to treat a specific condition of a current patient and a suggestion from a disease management advisor (DMA) for the current patient for the best medication to use based on the patient's current condition and past medical records.

FIG. 6 depicts an exemplary graphical user interface template having a plurality of standard physical exam questions with an exemplary question from a DMA integrated into a physician workflow to prompt a HCP to ask the exemplary question to a current patient for managing the current patient's condition.

FIG. 7 depicts an exemplary graphical user interface having an exemplary strategy integrated into a physician workflow for a HCP to write an electronic prescription for a medication from a list of medications to treat a specific condition of a current patient and a highlighted medication suggested by a DMA for the current patient as the best medication to use based on the patient's current condition, past medical records, and previous treatments.

FIG. 8 shows an exemplary graphical user interface to provide a way for disease management advisors to send information to healthcare professionals and have that information presented in appropriate way at an appropriate time.

FIG. 9 shows a flow chart depicting an exemplary organization of patient tasks before disease management functionality is added in an electronic medical record system workflow.

FIG. 10 shows a flow chart depicting an exemplary organization of patient tasks with disease management functionality integrated in an electronic medical record system workflow consistent with one aspect of the present invention may be incorporated into an electronic medical record (EMR) system.

FIG. 11 is a high-level block diagram of the architectural components of an exemplary healthcare EMR system including a plurality of healthcare provider workflow systems, a plurality of disease management advisor systems, and a plurality of patient systems indirectly communicating through a coordination server.

FIG. 12a is a high-level block diagram of the architectural components of an exemplary healthcare EMR system including a plurality of healthcare provider workflow systems, a plurality of disease management advisor systems, and a plurality of patient systems directly communicating through a network.

FIG. 12b is a high-level block diagram of the architectural components of an exemplary healthcare EMR system including a plurality of healthcare provider workflow systems, a plurality of disease management advisor systems, a plurality of patient systems, and a plurality of distribution systems directly communicating through a network.

FIG. 13 shows an exemplary graphical user interface of an electronic medical chart that may be used in an embodiment of the present invention to prompt a user to answer a set of questions from a disease management advisor for getting an approval to perform a procedure or test.

FIG. 14 shows an exemplary graphical user interface of an electronic medical chart that may be used in an embodiment of the present invention to select a procedure of a care plan.

FIG. 15 shows an exemplary graphical user interface of an electronic medical chart that may be used in an embodiment of the present invention by a disease management advisor to specify a set of questions that a health care professional may answer regarding approval to perform a procedure as a care plan.

FIG. 16 shows an exemplary graphical user interface of an electronic medical chart that may be used in an embodiment of the present invention by a health care professional to answer a set of questions that a disease management advisor may specify regarding approval to perform a procedure as a care plan.

FIG. 17 is a block diagram of the functional components of an exemplary interactive device adapted to provide an interface to a physician for facilitating a physical exam session.

FIG. 18 shows a top view of the interactive device of FIG. 17.

FIG. 19 depicts a block flow diagram of an exemplary method for use in the system of FIGS. 11, 12a and 12b.

### DETAILED DESCRIPTION

The invention is directed to a system and method for integrating disease management information into a physician workflow. More specifically, one or more third party disease management advisors may be integrated into the physician workflow. In one exemplary embodiment, an electronic medical record (EMR) device such as a wireless tablet, palm-sized computer, or desktop computer generally used by healthcare professionals (HCPs) is leveraged to integrate a plurality of disease management algorithms with the traditional EMR workflow of an electronic medical record (EMR) system. This integration provides a health care system with effective communication between a plurality of disease management advisors (DMAs) and HCPs. Furthermore, by extending the healthcare EMR system to a plurality of patients either in a clinic or outside of the clinic, it allows DMAs to communicate effectively with patients and helps them to communicate with HCPs. An exemplary embodiment of a healthcare EMR system consistent with the present invention is described to illustrate the functionality thereof by first describing how the healthcare EMR system facilitates communication between DMAs and HCPs by augmenting the traditional EMR workflow to include disease management functions within a physician workflow. Then, an exemplary embodiment of a healthcare EMR system and a method is described to illustrate how the physician workflow may be extended to include communication from patients to facilitate DMA-HCP-patient communication.

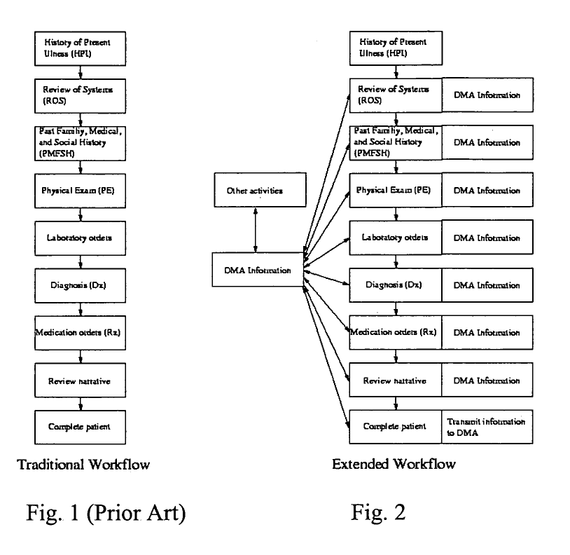

FIG. 1 is a flow chart depicting a prior art EMR workflow of traditional electronic medical records (EMRs) systems. As shown in FIG. 1, the traditional EMR workflow typically consists of several EMR tasks depicted as a set of steps including, History of Present Illness (HPI); Review of Systems (ROS); Past Family, Medical, and Social History (PMFSH); Physical Exam (PE); Laboratory Orders; Diagnosis (Dx); Medication Orders (Rx), Review Narrative; and Complete Patient Processing. Persons skilled in the art will appreciate that a variety of EMR tasks may be added, removed, combined, or split without changing the basic nature of this workflow. The arrows in the FIG. 1 illustrate a common EMR task order or flow, although EMR systems may allow other orders or allow random access or transition between one or more EMR tasks.

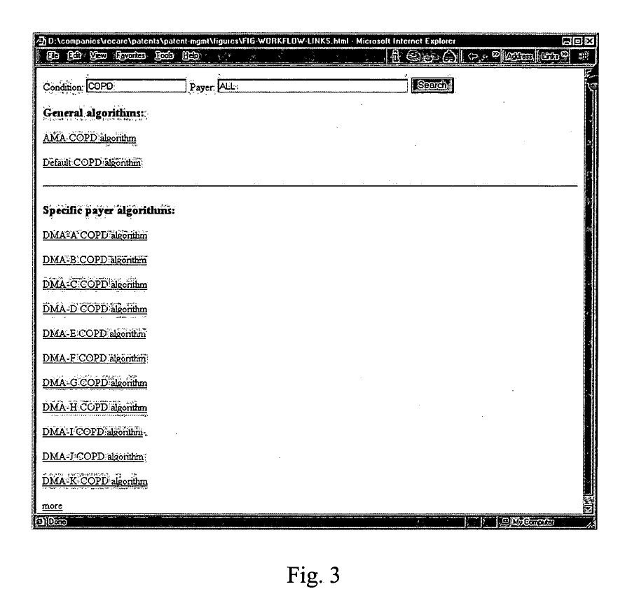

FIG. 2 is a flow chart showing an exemplary integration of disease management advisor (DMA) information and treatment algorithms into a physician/healthcare professional (HCP) workflow to provide an extended EMR workflow according to one embodiment of the present invention. For a physician-patient encounter, the extended EMR workflow adds the ability to view and interact with DMA information regarding a current patient by (a) providing a uniform method to a physician/HCP for accessing a plurality of treatment algorithms from a variety of DMAs; (b) providing the DMA information, finding questions, or potential orders associated with an EMR workflow task or EMR workflow element while specific traditional EMR tasks are being performed or specific EMR elements are displayed; (c) highlighting options corresponding to suggestions for questions, treatments, or tests in the available options in the extended EMR workflow; (d) providing a method for transmitting information gathered during the extended EMR workflow to the current patient's DMA; (e) transmitting statistical information that compares the physician's/HCP's compliance with the DMA rules for a DMA's patients to the physician/HCP; and (f) providing a method to transmit information related to the physician-patient encounter directly to the current patient.

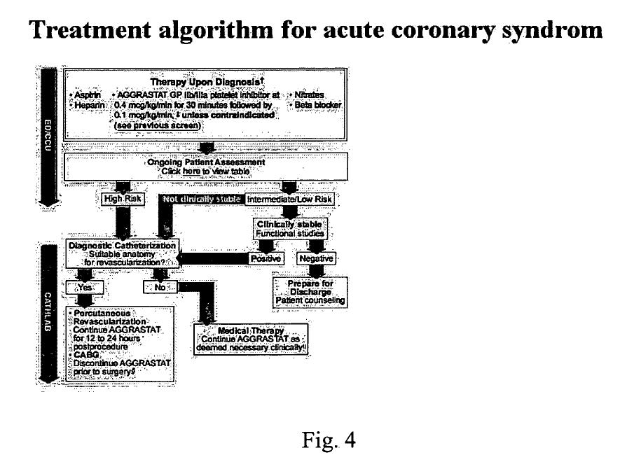

FIG. 3 illustrates an exemplary graphical user interface that may be used in an embodiment of the present invention for integrating DMA information from a plurality of disease management advisors (DMAs) and presenting that information to one or more healthcare professionals (HCPs). FIG. 3 illustrates an exemplary embodiment for step (a) illustrated with reference to FIG. 2. In an example application, an EMR workflow of a healthcare EMR system may integrate information from multiple DMAs and present such integrated information to a HCP within a single, uniform interface for searching, selecting, and/or communicating data. The depicted example application includes a plurality of DMA treatment algorithm links, and a plurality of fields including, a condition field and a payer field to communicate relevant data. Alternatively, a “Disease Management Advisor (DMA)” treatment algorithm field could be provided. Furthermore, by providing a single point of integration and communication for this type of information and/or data within a physician's workflow, (e.g., by implementing the system and the physician workflow on a wireless network-enabled tablet device), this information and/or data may be readily available to the HCP during the course of treating patients.

In one embodiment, the “Disease Management Advisor (DMA)” treatment algorithm field, the “condition” field, and the “payer” field may be automatically initialized from information stored for the current patient's medical record. Thus, by closely integrating this search with the EMR workflow in the physician workflow, the desired information can be made readily available to the HCP.

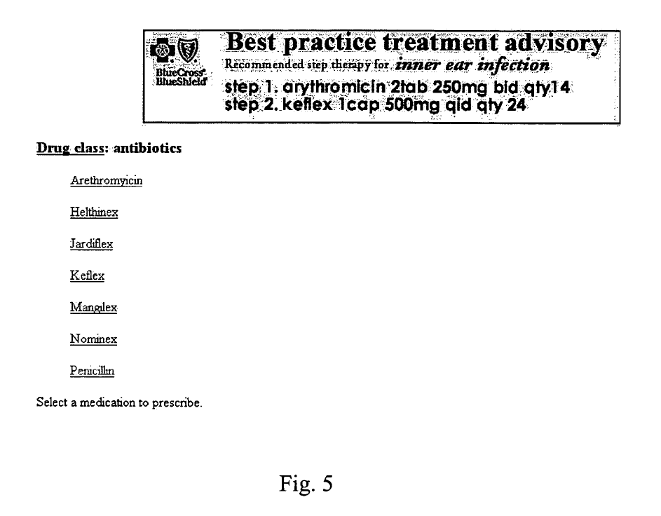

By activating a particular DMA treatment algorithm link of FIG. 3, detailed information could be displayed regarding the selected DMA treatment algorithm. FIG. 4 illustrates a display of an exemplary treatment algorithm for acute coronary syndrome consistent with one embodiment of the present invention.

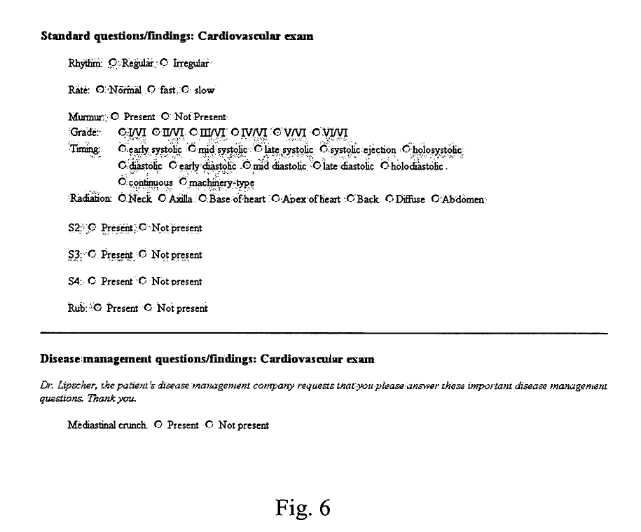

FIG. 5 depicts an exemplary graphical user interface having an exemplary strategy integrated into a physician workflow for a healthcare professional (HCP) to write an electronic prescription for a medication to treat a specific condition of a current patient and a suggestion from a disease management advisor (DMA) for the current patient for the best medication to use based on the patient's current condition and past medical records. Referring to FIG. 5 illustrates an exemplary implementation strategy according to step (b) of the extended EMR flow illustrated with reference to FIG. 2. In this exemplary implementation strategy, while a user such as a physician is using the extended EMR workflow of a healthcare EMR system within the physician workflow to write an electronic prescription for a medication to treat a specific condition, the healthcare EMR system provides through a workflow banner display the current patient's DMA's suggestion for the best medication to use based on the patient's current condition and past medical records. Similar displays may be included for other steps of FIG. 2 in the physician workflow, for example, suggestions for important questions to ask during the history of present illness (HPI) or review of systems (ROS), suggestions for examinations to undertake during the physical exam (PE), suggestions for laboratory tests to order on lab order screens, and so on. The displays may be informational only, or they may be “active” controls that the user may manipulate to input data into the EMR workflow of the healthcare EMR system. For example, as shown in FIG. 5, if the user selects the “step 1” area, the healthcare EMR system would jump to the prescription writing task and initialize a new prescription to the indicated parameters.

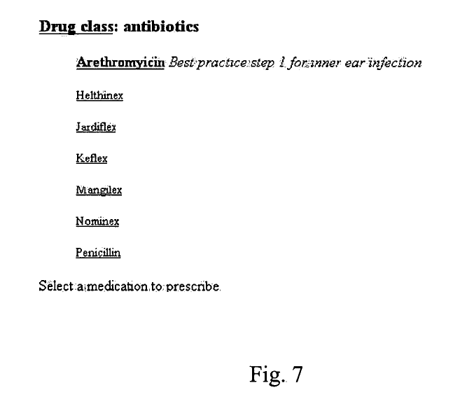

FIG. 6 depicts an exemplary graphical user interface for a workflow template to prompt a plurality of standard physical exam questions with an exemplary question from a DMA integrated into a physician workflow to prompt a HCP to ask the exemplary question to a current patient for managing the current patient's condition. As shown in FIG. 6, an exemplary implementation strategy according to step (b) of the extended EMR flow illustrated with reference to FIG. 2 is depicted. In this exemplary implementation strategy, physical exam (PE) questions specified by the DMA have been added to the standard physical exam template for the patient's condition. This prompts the HCP to ask questions he might not commonly ask that the DMA believes are important for managing the patient's condition effectively. Similar techniques can be used to provide relevant information in other steps of the extended EMR workflow within the physician workflow.

Combinations or variations of these implementation strategies may also be used. Accordingly, the functionality of FIGS. 5 and 6 can be readily combined. For example, the healthcare EMR system might display suggestions for tests to run or medications to prescribe in a manner similar to FIG. 5 but these displays may be “active” and allow input of findings or orders as in FIG. 6.

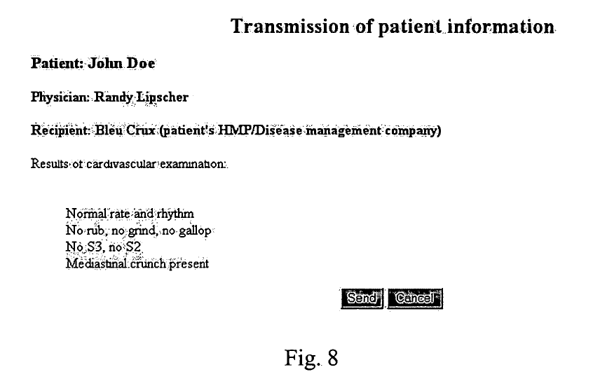

FIG. 7 depicts an exemplary graphical user interface having an exemplary implementation strategy integrated into a physician workflow for a HCP to write an electronic prescription for a medication from a list of medications to treat a specific condition of a current patient and a highlighted medication suggested by a DMA for the current patient as the best medication to use based on the patient's current condition, past medical records, and previous treatments. With reference to FIG. 7, the exemplary implementation strategy according to step (c) of the extended EMR flow illustrated with reference to FIG. 2 is depicted. In this exemplary implementation strategy, while a user such as a physician is using the extended EMR workflow of a healthcare EMR system within the physician workflow to write an electronic prescription for a medication to treat a specific condition, the system highlights the current patient's DMA's suggestion for the best medication to use based on the patient's current condition, past medical records, and previous treatments. In this example, since “step 1” therapy of FIG. 5 has not been tried yet, that therapy is highlighted. Similar techniques can be used to provide relevant information in other steps of the extended EMR workflow within the physician workflow.

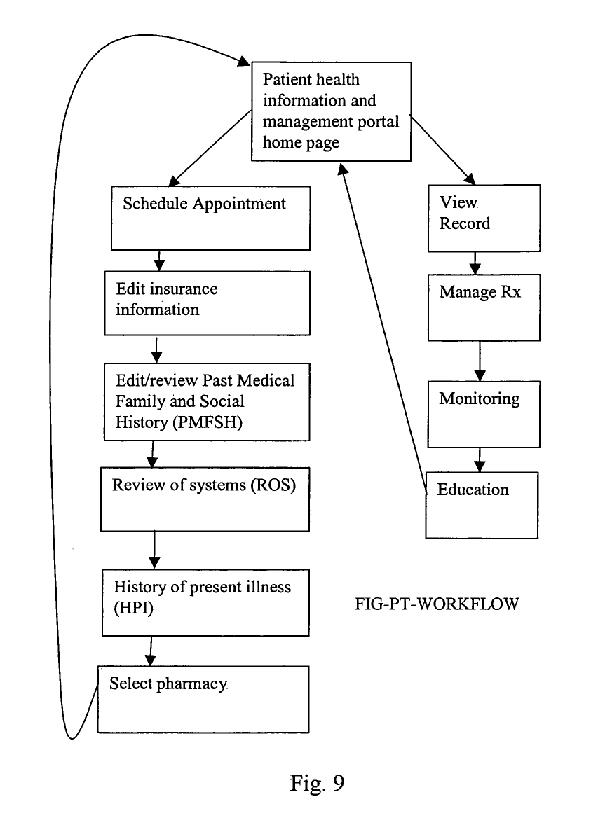

FIG. 8 shows an exemplary graphical user interface to provide a way for disease management advisors (DMAs) to send information to healthcare professionals (HCPs) and have that information presented in appropriate way at an appropriate time. Referring to FIG. 8, an exemplary implementation strategy according to step (d) of the extended EMR flow illustrated with reference to FIG. 2 is depicted. In addition to providing a way for DMAs to send information to HCPs and have that information presented in an appropriate way at an appropriate time, the healthcare EMR system also provides a channel to transmit information from HCP to DMA. For example, relevant medical findings and care plan orders from a patient-medical practitioner encounter are sent from the HCP to the patient's DMA. The subset of information to be sent (e.g. what medical findings or care plan orders are relevant for particular conditions), may be specified in the DMA's treatment algorithm or DMAs may make specific requests for specific information about specific patients. The information to be transmitted from the HCP or a clinic may include information entered by the HCP, information entered by other clinic staff (e.g., vital signs), information gathered directly by medical instrumentation devices and electronically placed in the medical record (e.g., results from an electronic network-enabled blood pressure cuff or EKG results), or information entered by the patient (e.g., history of present illness information entered in the waiting room). The information is transmitted from the patient's electronic medical record either by querying the physician or automatically sending the information at the completion of the patient-medical practitioner encounter as illustrated in FIG. 8. In both cases, the physician does not have to manually copy information from the medical record to be transmitted and only information relevant to DMA decisions and monitoring are sent.

According to an exemplary implementation strategy consistent with step (e) of the extended EMR flow illustrated with reference to FIG. 2, an appropriate statistical information that compares the physician's/HCP's compliance with the DMA rules for a DMA's patients may be transmitted to the physician/HCP. Likewise, in accordance with an exemplary implementation strategy consistent with step (e) of the extended EMR flow described with reference to FIG. 2, information related to the patient-medical practitioner encounter may be transmitted directly to the current patient. As described in the strategy for step (e) above, retrospective information about a physician's activities may be sent to DMAs so that DMAs can educate physicians about ways to improve their practice. For example, the healthcare EMR system can gather information about the treatments ordered by a particular HCP or group of HCPs for patients meeting particular criteria and send that information to a DMA. As an example, the healthcare EMR system can gather treatment information about all patients that have gone to a clinic complaining of ear pain or diagnosed with inner ear infections. The DMA could analyze this information to determine what fraction of the time the HCPs at the clinic follow their best practice guideline of prescribing amoxycillin for 7 days and only prescribing a stronger and more expensive antibiotic—keflex—if the patient returns with the same complaint and meets other specific criteria. Based on this information, the DMA could send a message to the clinic advising them how to improve their practice and including educational material on best practice in such situations. As described in strategy (f) above, the healthcare EMR system may provide an opportunity for a user such as a physician/HCP to send information recommended by the patient's DMA about a patient's condition to the patient.

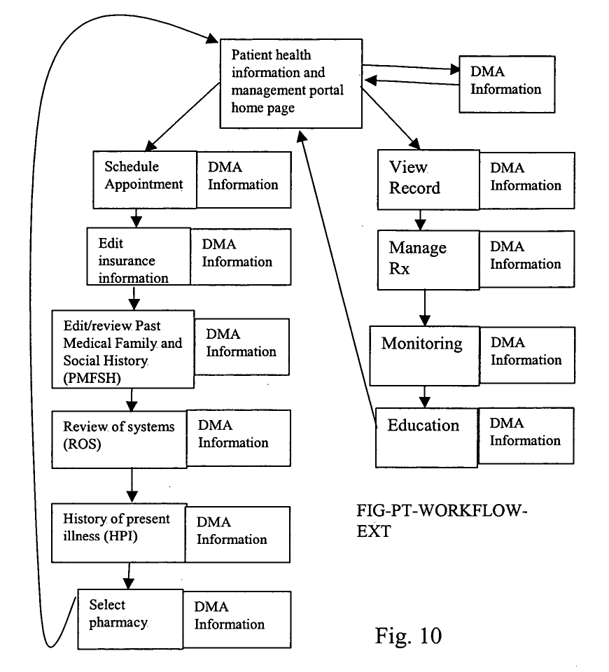

FIG. 9 shows a flow chart depicting an exemplary organization of patient tasks or steps before disease management functionality is added in an electronic medical record (EMR) system workflow. In one embodiment, patient tasks may comprise a plurality of pre-encounter tasks and a plurality of post-encounter tasks. For example, pre-encounter tasks may include, but not limited to, scheduling an appointment, in which patients make appointments with clinics; editing insurance info, in which patients update information about their insurance and contact information; and selecting a pharmacy, HPI, ROS, and PMFSH, in which patients enter current (HPI, ROS) and past (PMFSH) medical information. Such pre-encounter tasks may be conducted from the patient's home or work, from their portable or handheld machine, or from machines at the clinic. Likewise, key post-encounter tasks may include, but not limited to, viewing a record, in which the patient reviews the record of the visit, related educational material, or release instructions; managing Rx, in which patients control where their medication orders are sent; and monitoring, in which patients enter information about their health or activities (e.g., diet, body temperature, blood pressure, glucose readings, medications taken, mood). Other tasks could include, education, in which patients view educational material. It is to be understood that these depicted tasks and this organization are illustrative—other tasks or steps may be added, tasks may be removed, combined, or modified, and tasks may be moved or split among pre-encounter, post-encounter, and other times. In one embodiment, a patient health information home page is provided. Such patient health information home page allows direct access to each of the primary functions available to the patient.

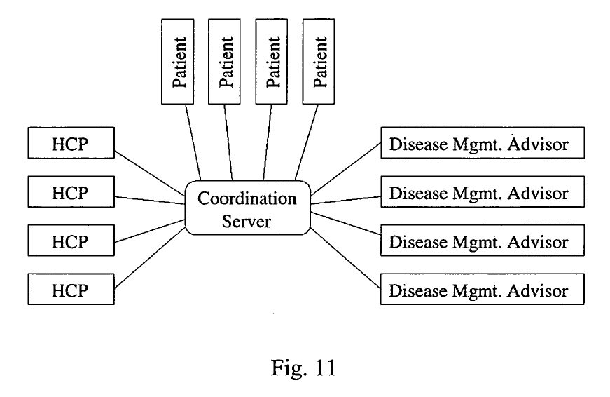

FIG. 10 shows a flow chart depicting an exemplary organization of patient tasks with disease management functionality integrated in an electronic medical record system workflow consistent with one aspect of the present invention. Referring to FIG. 10, which illustrates how disease management advisor (DMA) information may be integrated into a patient workflow and incorporated into an electronic medical record (EMR) system. This integration into the patient workflow may be accomplished using the same techniques described above for integrating DMA information into a physician workflow.

Examples of such integration include: the health care system can transmit disease management algorithms or educational material to the patient's computer; the healthcare EMR system can provide educational material and answer questions (using a human or an expert system) about the patient's disease management program; the healthcare EMR system can send DMA queries to the patient (‘did you take your medicine today?’} and send patient replies back to the DMA; the DMA can prompt the patient to enter monitoring information periodically (e.g., “enter your blood pressure daily”, “enter the meals you eat each day for a week”) or when specific criteria are met (e.g., “fill out this form whenever you get dizzy”); and the DMA can send instructions to the patient (e.g., “click here to schedule an appointment with your doctor”, “re-enter your blood pressure reading”).

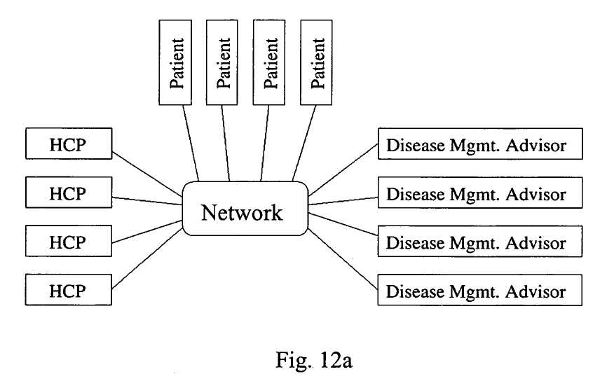

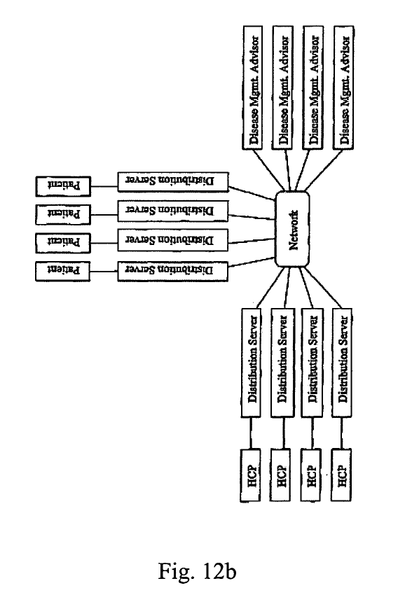

FIG. 11 is a high-level block diagram of the architectural components of an exemplary healthcare EMR system including a plurality of healthcare provider workflow systems, a plurality of disease management advisor systems, and a plurality of patient systems indirectly communicating through a coordination server. Likewise, FIG. 12a is a high-level block diagram of the architectural components of an exemplary healthcare EMR system including a plurality of healthcare provider workflow systems, a plurality of disease management advisor systems, and a plurality of patient systems directly communicating through a network. In one embodiment, there are many-to-many relationships among all of the categories of users: each HCP may interact with multiple patients and DMAs, each patient may have multiple HCPs and DMAs, and each DMA interacts with multiple HCPs and patients.

A variation of this system omits the patients' systems from an implementation. Although this limits the functionality of the system in various ways, much of the core functionality remains intact. To describe the inner workings of the system, a basic system EMR system that does not integrate disease management functionality is illustrated. Then the DMA functionality is added to such a system to provide a healthcare EMR system consistent with the present invention. An exemplary implementation of the internal components of a basic (non-DMA) system includes a traditional Electronic Medical Record (EMR), a template, a healthcare professional EMR interface, and a patient EMR interface.

According to conventional implementation of healthcare EMR systems, each Electronic Medical Record (EMR) typically comprises data elements that have information useful to the practice of medicine such as: one or more “patient records” with information about a patient such as patient identifying information (e.g., name, patient ID); patient demographic information (e.g., address, age, gender, marital status); and patient medical information. Typically, patient medical information can be divided into two parts: encounter record and patient status.

Each encounter record may refer to a list of “medical findings” and “care plan orders” made during a specific encounter between a patient and a HCP. An encounter would normally be a clinic visit, but it could also be a phone consultation, a consultant review of the patient chart, or the alike. A medical finding could be a medical observation, e.g., the answer to a history of present illness question, the answer to a review of systems question, the answer to a past family, medical and social history question, an observation from a physical exam, a result from a test, or a diagnosis. In general, a finding refers to an answer to a specific medical question such as “finding questions”. A care plan order comprises instructions or authorization to take some action, e.g., a prescription, release orders, staff orders, an order for a test, or counseling information to be sent to the patient. For example, the term “potential order” refers to a possible instruction or authorization for a specific action that has not been selected by the HCP. Patient status provides patient status information, which reflects a summary of the current relevant medical findings for a patient, e.g., active problems, current medications, and/or allergies.

A template specifies a subset of the “patient medical information” that is clinically relevant under some condition. The specified subset may include (a) medical findings and care plan orders from previous encounters, (b) patient status, and (c) medical findings/care plan orders and finding questions/potential orders for the current encounter. In other words, a template lists “questions the HCP might want to ask” and/or “orders the HCP might want to make”—whether or not they have yet been asked or ordered—along with selections made so far. However, not all questions on a template for an encounter may be asked or orders made; e.g., template could list “top 10 tests to order for patient complaining of chest pain”; but in a typical encounter, an HCP may select one of those ten to order based on their physical examination.

Commonly, templates are organized on a per-complaint basis. For example, the templates may take the form of a template for “chest pain”, a template for “sore throat”, or the alike. For clarity of the present exposition, a similar organization is assumed in the rest of the description except where noted. However, as persons skilled in the art will recognize other possible organizations are possible such as templates can be dynamically generated by rules based or expert systems or based on the current set of findings. Alternatively, templates can be configured as Boolean sets of rules (“if A is selected then ask B and order C”).

A HCP EMR interface generally provides a method for viewing/editing information made available to a HCP. For example, for viewing and editing which elements from a template are marked as selected/not selected and for viewing medical findings/care plan orders from past encounters or viewing/editing patient status information. Again for clarity purposes, it is assumed that the HCP EMR interface and templates are each divided into “tasks or steps,” including, but not limited to, History of Present Illness (HPI), Physical Exam (PE), Diagnosis (Dx), Prescription Writing or Medication Orders (Rx), Past Family Medical and Social History (PMFSH), and the alike. For example, the HPI task in the interface will display the elements of the template denoted as part of the HPI.

To a patient, a patient EMR interface generally provides a method for viewing and editing which elements from a template are marked as selected/not selected and for viewing medical findings/care plan orders from past encounters or viewing/editing patient status information.

Patient tasks could include such activities as patient enter/view insurance info, patient enter/view past medical family social history, patient enter/view demographic information, patient enter/view chief complaint, patient enter/view allergies, patient update/view current medicines, patient answer questions, patient enter HPI, patient enter ROS, patient view medical record, patient enter/view compliance monitoring, and patient enter/view condition monitoring. While it is convenient to think of the patient's EMR interface to the healthcare EMR system as a simple variation of the HCP's view of the system where the patient simply conducts different tasks and therefore sees (and controls) different subsets of information, typically the patient tasks will provide a more restricted view and fewer available options than the HCP tasks.

In an exemplary embodiment, integration of disease management to a traditional EMR workflow is devised through adding to a physician workflow one or more treatment algorithms from one or more disease management advisors (DMAs). Each DMA specifies one or more treatment algorithms. In an exemplary implementation, each treatment algorithm comprises two parts: a rules based selection and an algorithm template.

Rules based selection determines if a particular algorithm is relevant to the current patient. The following method illustrates a simple rules based selection embodiment: Each treatment algorithm has a selection function, which is a function of patient record (e.g., past findings, current findings, patient status and other patient information)

```
if(algorithm, selection(patientRecord) == true)
  then algorithm is relevant
if(algorithm.selection(patient-record) == false)
  then algorithm is not relevant
```

In addition, each treatment algorithm is associated with a particular DMA, and the healthcare EMR system only considers treatment algorithms indicated by the patient's own DMA and ignores others.

```
if(patient-record.patientsDMA != algorithm.DMA) {
  algorithm is not relevant
else
  algorithm is relevant
```

In addition, some treatment algorithms' DMA fields may be set to the special value ALL, which indicates that they may be relevant to a patient regardless of the patient's DMA. Examples of this are when a treatment algorithm is recommended by a best-practice authority such as the American Medical Association. So, the procedure to select a treatment algorithm is to select the algorithm(s) (if any) where

algorithm.selection(patientRecord) is true
AND
(patient-record.patientsDMA == algorithm.DMA
OR
algorithm.DMA == ALL)

Also note that some patients may have multiple DMAs and some treatment algorithms may be associated with multiple DMAs. In such cases if any patient DMA matches any treatment algorithm DMA, then selection is activated.

Each treatment algorithm may also comprise a priority, which allows the healthcare EMR system to rank conflicting elements for display. For example, in one embodiment if two different treatment algorithms both want to display a message in the banner display area of a screen, the one with higher priority is selected by the system. Conversely, in one embodiment all activated elements are displayed together but their method of display depends on their priority (e.g., different highlighting or order). For instance, in one embodiment, at least one priority level is designated as “critical” and is always displayed, even if multiple “critical” items are active.

An algorithm template lists treatment algorithm elements—information, finding questions, and potential orders—that are relevant when rules match patient. In one embodiment, each treatment algorithm element comprises (a) content (comprising (i) a type (e.g., is the element information, finding question, prerequisite questions, or a potential order; e.g., a new question, the directive to highlight a question, the directive to issue an order, the directive to display information, the directive to transmit information to the DMA), (ii) an identifier for the information to be displayed or highlighted, finding question to be asked or highlighted, or order to be displayed, issued, or highlighted; in one embodiment this identifier is the element's unique ID in the EMR) or the definition of a new piece of information, question, or order) (b) a set of conditions for when the element should be activated (e.g., Boolean functions of patient findings or patient status or condition or a frequency), (c) and a location for where and when the element should be displayed (e.g., a task identifier that indicates that the element should be displayed when the specified task is active and/or a finding or potential order identifier that indicates that the element should be displayed when the specified finding or potential order is displayed by the base template.)

An element may contain multiple type/identifier pairs if multiple elements are to be displayed under the same conditions and in the same location. Likewise, a set of elements may specify conditions and locations that refer to one another in order to create a logical flow between treatment algorithm elements (e.g., if user answers X then ask Y)

A question or set of questions may be specified as a prerequisite question for an item. A prerequisite question specifies (a) the guarded item as an identifier of a order element (e.g., test or medication) in the EMR, (b) zero or more questions to be asked (some of these questions may simply refer, and (c) a Boolean function across the results of the questions and other data fields in the patient's EMR. A prerequisite question is used to obtain approval of an order. In one embodiment, the user interface replaces the element with that order with an element that activates the prerequisite question or questions and, upon completion, either activates the order (if the prerequisite's Boolean function evaluates to true) or provides a link to cancel the order or contact the DMA (if the Boolean function evaluates to false.)

A healthcare EMR system may also prioritize questions/orders in the algorithm template or provide other presentation information. The identifiers may refer to information, questions, or orders in different formats supported by the EMR including graphical images, text, HTML, XML, or script or machine code that describes how to render the specified information/question/order.

Given these basic components, in an exemplary implementation, a healthcare EMR system may proceeds as follows. In step 1, two or more DMA entities enter algorithms into the system. In step 2, a user selects a patient to work with. In step 3, each time the user activates a task, changes the display, or updates information about the current patient, a) the healthcare EMR system displays the current task's portion of the default EMR template (selected based on the chief complaint and/or findings) and/or b) if any DMA algorithm selection rule evaluates to true the system selects the elements to be displayed for the current task/set of EMR template elements according to the rules for the selected DMA algorithm elements. The healthcare EMR system displays these elements according to the rules specified in the elements by (i) augmenting the EMR template's information/questions/potential orders with the DMA template element's selected information/questions/potential orders (as shown in FIGS. 5 and 6) and (ii) highlighting orders from the EMR template as specified by the selected DMA template elements as shown in FIG. 7.

In step 4, the healthcare EMR system provides the option for the user to explicitly view DMA information (as shown in FIG. 3). In step 5, when the user completes a task or finishes an encounter, the system sends the findings and orders that match the selected algorithm's template to the DMA. Optionally, the healthcare EMR system may display a confirmation screen before doing this (e.g., as shown in FIG. 8).

A DMA may wish to provide guidance to an HCP, but some HCPs will be resistant to techniques that add additional steps to the documentation process or that “pre-select” elements without the HCP's explicit approval. The methods and systems of the present invention generally provide a means for resolving this dilemma in two illustrated approaches.

In first approach, rather than add steps or information, the DMA may “highlight” elements already present in the HCP's workflow. This highlighting may take the form of a visual cue (e.g., color, font, size, and icon) directing attention to an element that should be selected or a question that should be answered by the HCP. In one embodiment different highlights reflect different levels of importance of an element (e.g., blue for “advisory” and red for “critical.”) In another embodiment, this highlighting may take the form of ordering a list of options to place highlighted elements in a preferred location such as the top of a list or a separate list or menu of preferred choices.

In second approach, the DMA may highlight a set of elements and provide the additional option to “select all highlighted elements”. If this option is selected, all of the highlighted elements are automatically selected as if the HCP selected each highlighted element. This approach provides, for example, a method for the HCP to select a battery of tests suggested by a DMA for a particular situation.

Using this framework a variety of embodiment may be contemplated to facilitate communication among HCPs, DMAs, and patients. For a patient status prompting and maintenance based implementation, in one realization, the DMA, HCP, and patients use the system to regularly document patient status information such as smoking status or alcohol consumption and to transmit this information to the DMA. In an alternate realization, a DMA (DMA-A) specifies that it would like HCPs to ask all patients belonging to DMA-A about their smoking status at least once per year. Then, when the HCP activates the PMFSH task, the healthcare EMR system prompts that HCP for patient smoking status if it has been more than one year since the status was last entered. After the HCP enters that information, the system transmits that update (along with the patient name, patient ID, HCP ID, and date of visit) to the DMA. In another realization, such an algorithm is expressed with a record comprising several fields including:

```
DMA = DMA-A;
SELECTION = ALWAYS;
TASK = PMFSH;
CONDITION = ALWAYS;
FREQUENCY = 1 year;
CONTENT = QUESTION ID_OF_SMOKING_STATUS_QUESTION,
            ORDER TRANSMIT_TO_DMA
            ID_OF_SMOKING_STATUS_QUESTION,
```

Other variations are possible. For example, rather than adding a new question for smoking status, one realization indicates that the smoking status question should be highlighted if it has not been answered in the last year. In one realization, such an algorithm could be expressed:

```
DMA = DMA-A;
SELECTION = ALWAYS;
TASK = PMFSH;
FREQUENCY = 1 year;
CONTENT = HIGHLIGHT ID_OF_SMOKING_STATUS_QUES-
            TION, ORDER TRANSMIT_TO_DMA
            ID_OF_SMOKING_STATUS_QUESTION,
```

In yet another realization, the DMA indicates that the healthcare ENR system should ask the patient about smoking status when the patient answers Patient-PMFSH questions, and that the healthcare EMR system should then transmit the information to the DMA.

```
DMA = DMA-A;
SELECTION = ALWAYS;
TASK = PATIENT-PMFSH;
CONTENT = QUESTION ID_OF_SMOKING_STATUS_QUESTION,
            ORDER TRANSMIT_TO_DMA
            ID_OF_SMOKING_STATUS_QUESTION,
```

These smoking status questions are illustrative of a broad range of questions about patient status related to the PMFSH that could be implemented using the system. Other topics of potential interest include: family heart disease history, weight, family cancer history, family glaucoma history, diet habits, exercise habits, and alcohol consumption.

For a preventive care and immunization based implementation, in one realization, the DMA, HCP, and patients use the system to regularly perform and document preventive care procedures according to the practices recommended by the DMA. For example, a DMA (which is referred to as DMA-A) may wish its male patients over the age of 40 to receive a prostate exam once every year. The DMA could specify this as a treatment algorithm and transmit the treatment algorithm to the healthcare EMR system. Then, when the HCP is accessing the physical exam task, if the current patient meets those criteria, the healthcare EMR system prompts the user to perform the exam (for example, by displaying a banner graphic indicating that a yearly prostate exam is recommended for the current patient). Finally, when the HCP completes the encounter, the healthcare EMR system includes the results of the prostate exam in the report sent to the DMA. In one realization, such a rule is expressed as follows:

```
DMA = DMA-A;
SELECTION = ALWAYS;
TASK = PE;
FREQUENCY = 1 year;
CONDITION = GENDER = M & AGE 40;
CONTENT = QUESTION ID_OF_PROSTATE_EXAM_QUESTION,
            ORDER TRANSMIT_TO_DMA
            ID_OF_PROSTATE_EXAM_QUESTION,
```

A second DMA (which is referred to as DMA-B) might have different rules for when it recommends (and will reimburse) a prostate exam. It might recommend a prostate exam for male patients over the age of 30 but with a frequency of once every two years. In one realization, such an algorithm is expressed as follows:

```
DMA = DMA-B;
SELECTION = ALWAYS;
TASK = PE;
FREQUENCY = 2 year;
CONDITION = GENDER = M & AGE 30;
CONTENT = QUESTION ID_OF_PROSTATE_EXAM_QUESTION,
            ORDER TRANSMIT_TO_DMA
            ID_OF_PROSTATE_EXAM_QUESTION,
```

Another example of using the healthcare EMR system to prompt users for preventive care is a DMA that wishes to prompt users for a bi-annual physical exam. In this case, any time a patient logs into the system (e.g., to review their health information from a health portal linked to the EMR) if it has been more than 2 years since the patient's previous physical exam, then the patient is prompted to make an appointment with his or her doctor. This prompt could take the form of a banner display linked to a web page for making an appointment at the patient's doctor's clinic. In one realization, such an algorithm is expressed as follows:

DMA = DMA-A;
SELECTION = ALWAYS;
TASK = PATIENT-*;
FREQUENCY = 2 year;
CONTENT = INFO ID_OF_PHYSICAL_EXAM_RE-
MINDER_WITH_LINK

Preventive care prompting may also be driven by specific data elements entered. For example, some DMAs may wish to remind HCPs to perform a yearly foot exam on diabetes patients and to further prompt the HCP to perform such an exam when working with a diabetic patient who has not had a recent foot exam. The DMAs would specify this rule, and the healthcare EMR system could prompt the physician during the physical exam of such a patient. In one realization, such an algorithm is expressed as follows:

DMA = DMA-A;
SELECTION = ALWAYS;
TASK = PE;
FREQUENCY = 1 year;
CONDITION = DIABETES_STATUS_ID = TRUE;
CONTENT = QUESTION ID_OF_DIA-
BETIC_FOOT_EXAM_QUESTION

In another embodiment, a DMA may wish to modify only a particular template. For example, DMA-A may wish to augment the template for “fracture” to, encourage calcium supplements for middle-aged or older females. In one realization, such an algorithm is expressed as follows:

DMA = DMA-A;
SELECTION = FRACTURE_TEMPLATE;
TASK = ANY;
FREQUENCY = ALWAYS;
PRIORITY = LOW;
CONDITION = AGE 40 && GENDER = F;
CONTENT = INFORMATION TEXT:“A recent
AMA study recommends supplemental calcium for most
females over 40”

In one embodiment, that text information is replaced by a JPEG or GIF image. In one embodiment, the text information is hyper-linked to a more detailed discussion. Other preventive care prompting examples include prompting the HCP to order a mammogram for patients that have not had one recently, prompting the HCP to order a cholesterol test if the patient is overweight and has not had such a test recently, prompting the HCP to prescribe a smoking cessation product if the patient's smoking status is TRUE and the patient is not currently taking that medication, and so on.

For an exemplary patient education task implementation, in one realization the DMA, HCP, and patients use the healthcare EMR system to transmit educational material to patients. In another realization, a DMA may develop a set of smoking cessation information that they wish to transmit to their smoking patients. The DMA wishes to prompt the HCP to authorize sending that information when the patient visits the HCP when the HCP is engaged in PMFSH task for the patient and to send that information only if the HCP approves. In one embodiment, such an algorithm is expressed with the element:

DMA = DMA-A;
SELECTION = ALWAYS;
TASK = PMFSH;
FREQUENCY = 30 days;
CONDITION =
ID_OF_SMOKING_STATUS_QUESTION = true;
CONTENT = QUESTION
ID_OF_SEND_SMOKING_CESSATION_INFO_PROMPT,

ORDER TRANSMIT_TO_DMA
ID_OF_SEND_SMOKING_CESSATION_INFO_PROMPT

Similar rules could allow a DMA to communicate diet, exercise, diabetes management, pregnancy, and other information. In one realization, a DMA may wish to send educational material to its patients while the patients are using the healthcare EMR system to enter pre-clinical encounter information. For example, in one embodiment, the healthcare EMR system provides Patient-PMFSH review, Patient-HPI, and patient-INFO tasks that allow patients to fill in updates to their past medical, family and social history, to enter the history of their present illness and to view selected health information while they wait in the clinic waiting room. For example, if a patient indicates that they have a fever, the DMA may wish to display rules for distinguishing a cold from a fever to the patient while they wait. In one embodiment, such an algorithm is expressed with the element:

DMA = DMA-A;
SELECTION = COLD_OR_FLU_TEMPLATE;
TASK = Patient-INFO;
CONDITION = ID_OF_FEVER_QUESTION = true;
CONTENT = INFO
ID_OF_DMA_A_COLD_V_FLU_INFO_SHEET

Similar arrangements allow a DMA to provide smoking cessation, diet, exercise, cancer screening, heart disease and similar information to appropriate patients while those patients are in a clinic waiting room or at home filling out pre-clinical information. Such an arrangement would also allow a DMA to inform a patient about general procedures or features of the DMA, such as “Call 1-800-my-nurse for answers to any medical question,” or “Go to http://www.DMA-A.com for answers to your health questions” or “Emergency room visits are only reimbursable if DMA-A is notified within 48 hours of the visit” and so on.

In one realization, a DMA may wish to prompt HCPs to counsel a patient about a particular problem and may provide specific counseling information. The DMA could specify an algorithm that reminds the HCP to do so during the encounter. In one embodiment, such an algorithm is expressed with the element:

DMA = DMA-A;
SELECTION = ALWAYS;
TASK = PMFSH;
CONDITION = Body_mass_Index 28 &
ID_PATIENT_REPORT_EXERCISE DMA-

A_DIET_AND_EXERCISE_INFO

Then, when the HCP accesses the PMFSH task for a patient with a body mass index greater than 28 and whom reports exercising less than once per week, the healthcare EMR system with the extended EMR flow will prompt the HCP with DMA-A's diet and exercise lecture to be delivered by the HCP to the patient.

For resource utilization based embodiments, in one embodiment, the healthcare EMR system provides methods whereby DMAs can offer guidance to HCPs to improve resource utilization to reduce costs and improve quality of care. For example, the DMA may specify that certain medications, such as those on a “formulary list”, are preferred to others. This can be accomplished, for example, by highlighting on-formulary medications, highlighting off-formulary medications, or adding a “banner” of information for medication X that is displayed when medication Y is displayed.

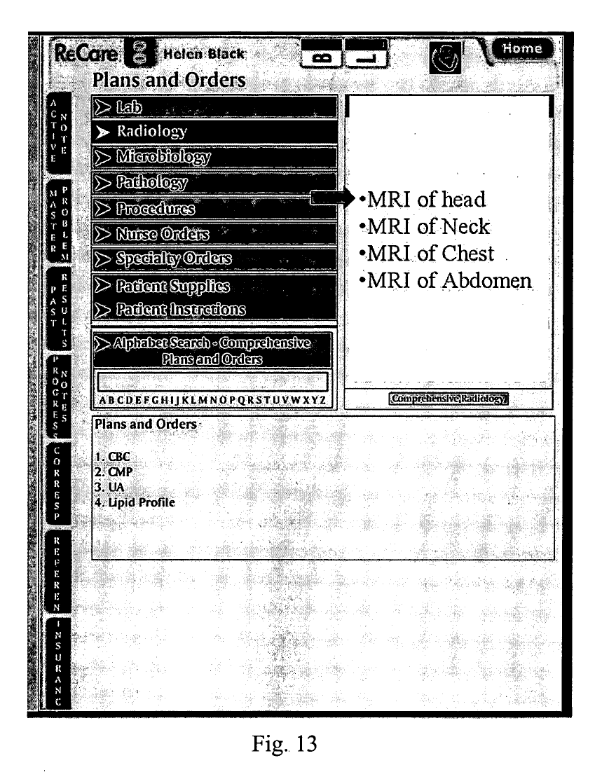

For example, the DMA may specify a set of questions that the HCP should answer to get approval to prescribe a given “off-formulary” medication. In one embodiment, this approval process is accomplished by specifying a question with ID OFF-FORMULARY-DMA-1-MEDICATION-X for approval of medication X by DMA-1 that is displayed when medication X is displayed or when medication X is selected. In another embodiment, if the set of questions is long, this approval process is accomplished by specifying one initial question that is displayed when medication X is displayed and specifying several additional questions that are asked if the initial question is answered and matches some condition. In another embodiment, this set of questions is specified as a “prerequisite question” as described above. For example, a similar methodology may be used by a DMA to specify a set of questions that the HCP should answer to get approval to perform a procedure or test. This process is illustrated in FIGS. 13 through 16.

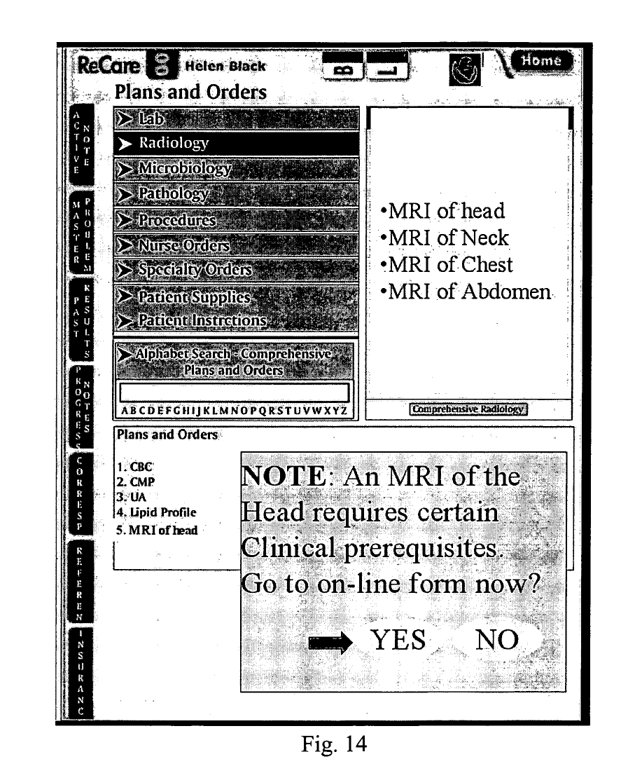

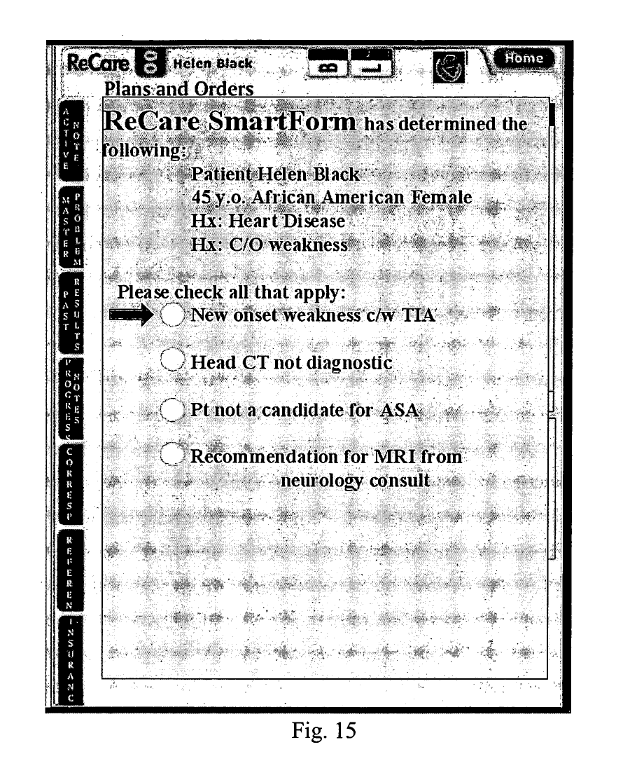

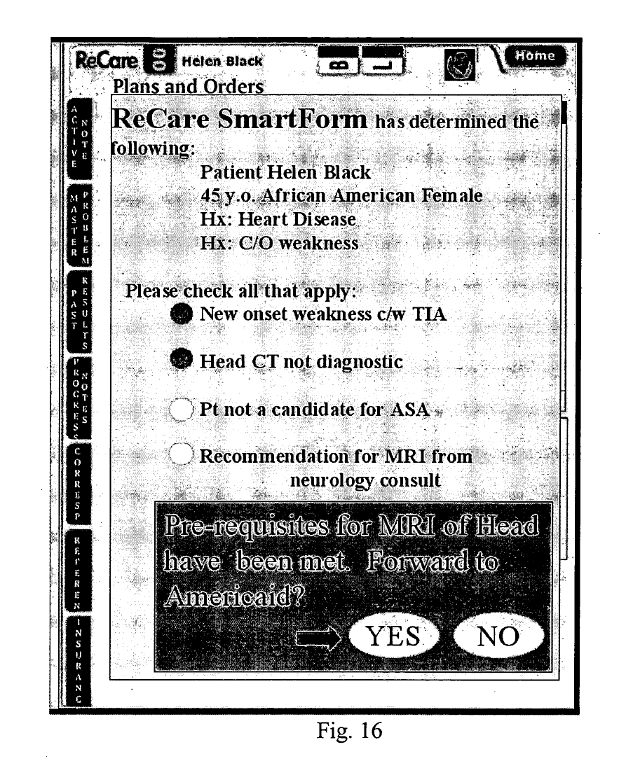

FIG. 13 shows an exemplary graphical user interface of an electronic medical chart that may be used in an embodiment of the present invention to prompt a user to answer a set of questions from a disease management advisor for getting an approval to perform a procedure or test. FIG. 14 shows an exemplary graphical user interface of an electronic medical chart that may be used in an embodiment of the present invention to select a procedure of a care plan. FIG. 15 shows an exemplary graphical user interface of an electronic medical chart that may be used in an embodiment of the present invention by a disease management advisor to specify a set of questions that a health care professional may answer regarding approval to perform a procedure as a care plan. FIG. 16 shows an exemplary graphical user interface of an electronic medical chart that may be used in an embodiment of the present invention by a health care professional to answer a set of questions that a disease management advisor may specify regarding approval to perform a procedure as a care plan.

In another exemplary implementation, a DMA, such as a prescription benefits management company, health maintenance organization, or insurance company, would use the system to direct patients to use specific preferred pharmacies to fill their prescriptions. For example, this implementation would specify that for patients associated with a particular DMA, during a patient pre-encounter task, such as patient-PMFSH or patient-review-insurance-information, that the system should insert content to prompt the patient to select a pharmacy to which orders will be transmitted.

In another exemplary implementation, a DMA such as a pharmacy company would use the system to encourage patients to use a specific preferred pharmacy. For example, this system would specify that for all patients, during a patient select-pharmacy task, that the system should insert content such as a banner ad or highlighted content such as a selectable input element to encourage the patient to select the specified pharmacy.

In another exemplary implementation, a DMA such as an HMO would use the system to communicate with or guide an HCP who is referring a patient to a different HCP or specialist HCP such as a surgeon or dermatologist. In one such implementation, a DMA would specify that when an HCP is working on the SPECIALIST_REFERRAL task and the current patient is associated with the DMA, the system should insert content such as a list of DMA-approved specialists or highlight DMA-approved specialists on the default list. In another such implementation, a DMA would specify that when an HCP is working on the CARDIAC_SPECIALIST_REFERRAL task and the current patient is associated with the DMA, the system should insert content such as a list of DMA-approved cardiac specialists or highlight DMA-approved cardiac specialists on the default list. In another exemplary implementation, a DMA would specify that when an HCP is working on the SPECIALIST_REFERRAL task and the current patient is associated with the DMA and under the condition that the current encounter record includes diagnosis of congestive heart disease, the system should insert content such as a list of DMA-approved cardiac specialists or highlight DMA-approved cardiac specialists on the default list.

In another exemplary implementation, a DMA such as an HMO or benefits management company would use the system to communicate with or guide an HCP who is ordering a laboratory test or procedure. In one such implementation, a DMA would specify that when an HCP is working on the ORDER_LABS task and the current patient is associated with the DMA, the system should insert content such as a list of DMA-approved labs or highlight DMA-approved labs on the default list. In another such implementation, a DMA would specify that when an HCP is working on the ORDER_CBC_LABS task and the current patient is associated with the DMA, the system should insert content such as a list of DMA-approved lab for CBC tests or highlight DMA-approved labs for CBC tests on the default list.

In another exemplary implementation, a DMA would specify to the system that it wishes to receive information gathered by HCPs during an encounter with patients associated with the DMA. In one such implementation, a DMA would specify that for patients associated with the DMA, the system should insert in the FINISH_ENCOUNTER task content comprising a prompt for the HCP to transmit all findings from the encounter to that DMA. In one implementation, this content element includes a text message requesting that the HCP transmit the information, a set of hidden elements corresponding to the patient's ID and all findings by the HCP about the patient for the current encounter, and an activation button where activating the button causes the hidden elements to be transmitted to the DMA.

In another exemplary implementation, a DMA would specify to the system that it wishes to enroll “high risk” patients such as diabetics in specific disease management programs. In one such implementation, a DMA would specify that for patients associated with the DMA, the system should insert in the FINISH_ENCOUNTER task content comprising a prompt for the HCP to transmit the patient's name, ID, and diagnosis to that DMA on the condition that the patient is diagnosed with diabetes. In one implementation, this content element includes a text message requesting that the HCP transmit the information, a set of hidden elements corresponding the patient's ID and diagnosis, and an activation button where activating the button causes the hidden elements to be transmitted to the DMA. In another exemplary implementation, a DMA such as a medical research institute would specify to the system that it wishes to enroll patients meeting criteria specified by the DMA in specific medical studies such as medication trials. In one such implementation, a DMA would specify that for all patients, on the condition that the patient's encounter record has fields meeting specified Boolean tests (e.g., age>18 AND gender=F AND smoking=no AND diagnosis=Flu) the system should insert in the FINISH_ENCOUNTER task content comprising a prompt for the HCP to transmit the patient's name, ID, and diagnosis to that DMA in order to enroll the patient in a study. In one implementation, this content element includes a text message describing the study and requesting that the HCP transmit the information, a set of hidden elements corresponding the patient's ID, demographic and contact information, and diagnosis, and an activation button where activating the button causes the hidden elements to be transmitted to the DMA.

In another exemplary implementation, a DMA such as an HMO or prescription benefits management company would use the system to communicate with or guide an HCP who is ordering a medication to encourage the physician to select an appropriate medication based on data-driven medicine such as step therapy. In one such implementation, a DMA would specify that when an HCP is working on the Rx task and the current patient is associated with the DMA, and on the condition that the diagnosis matches a specific diagnosis (e.g., diagnosis=ear infection) and that the patient's medical history does not indicate that a first-line antibiotic has been prescribed for the patient (e.g., medications during past 7 days does not include amoxycillin or tetracycline) and that the current encounter record indicates that a powerful or expensive antibiotic has been prescribed (e.g., new medications includes keflex), the system should insert content such as banner display or message reminding the physician that a specified first-line antibiotic should generally be prescribed before a more powerful or more expensive antibiotic is used (e.g., “For patients with ear infection, the recommended step therapy begins with a 1 week course of amoxycillin or tetracycline. Keflex should generally be prescribed only if the patient does not respond to these initial medications.”)

Further, the healthcare workflow may be altered in accordance with the medical diagnostic and treatment algorithm. The modification may take various forms. These forms may include a banner, a highlighted option, a question, information, a warning, an electronic form, an action such as a sending of a message to a user or other, an input method for executing orders, an auditory notification, a tactile notification, a change in workflow order, an addition of one or more steps to the workflow process, adding a hyperlink, notification of a drug trial, recommendation of step therapy, a question for the HCP to ask the patient, a question for the patient, a request for a test or observation, a prompt to do periodic preventative care or immunization, a input to activate sending educational material to the HCP or patient, a prompt to send information to the DMA, a highlighting or display of a subset of possible medications, a highlighting or display of a subset of medical specialists for referral, a highlighting or displaying of a subset of laboratories to which orders may be sent, a highlighting or display of a subset of pharmacies to which medication orders may be sent, a display of a form for approval of a medication, a display of a form for approval of a test, a prompt to or automatic enrollment of a patient in a disease management program.

In an exemplary implementation, the system also comprises at least one or a plurality of Algorithm-Input servers. Each Algorithm-Input server provides an interface whereby a DMA may create, update, edit, and delete their disease management algorithms. In an exemplary implementation, an Algorithm-Input server stores each DMA's algorithms locally. In another exemplary implementation, the Algorithm-Input server stores each DMA's algorithms at the Coordination Server. In an exemplary implementation, a single Algorithm-Input server provides an interface to a plurality of DMAs and enforces access control to ensure that DMAs access only algorithms they own or have been granted permission to access. In another exemplary implementation, a plurality of Algorithm-Input servers each provide access to a specific subset of DMAs. For example, each DMA may use an Algorithm-Input server located locally at the DMA.

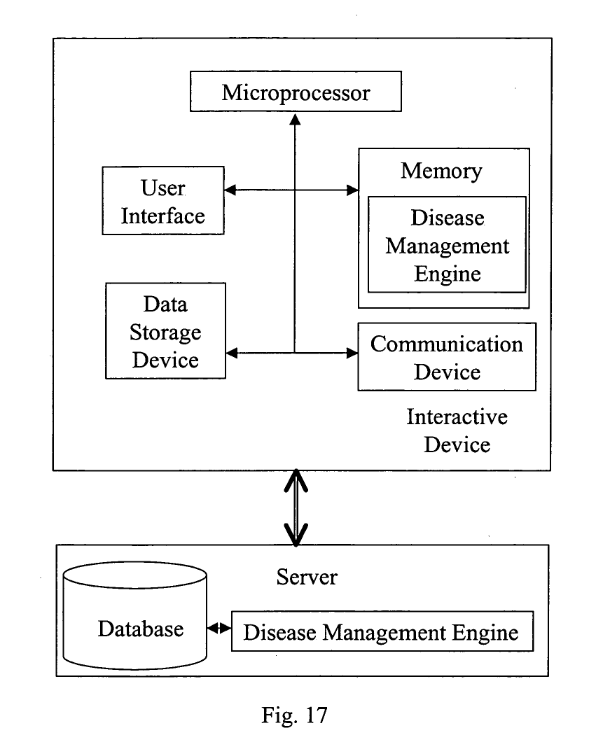

FIG. 17 is a block diagram of the functional components of an exemplary interactive device adapted to provide an interface to a physician for facilitating a physical exam session. Interactive device includes a microprocessor executing a computer program stored at least in part in a read only memory (ROM) and carrying out many of the steps of the present invention. Interactive device includes a communications device for communicating with a server on, which may reside additional portions of the computer program and data used in carrying out the invention. Interactive device also uses a random access memory (RAM) for temporary information storage.

Interactive device also includes at least one output interface and associated circuitry for communicating information to a user such as a physician, as well as one or more input interfaces, such as a touch sensitive screen and a microphone, with associate circuitry for receiving information from the physician. Output interface can provide information to the physician visually, audibly, or in any combination of ways. Input interfaces can allow input in any number of ways, such as by a touch screen, keyboard, voice capture, voice data recognition, voice command recognition, handwriting image capture, cognitive handwriting recognition, or any other way or combination of ways of receiving communications to the physician. Communication device or a different communication device can optionally support data ports for connection external devices, such as thermometers or blood pressure measurement devices.

The interactive device could comprise, for example, a desktop, laptop, tablet, or other type of computer. The preferred embodiment of interactive device may change as technology evolves. The components that comprise interactive device do not need to be physically incorporated into a single unit. For example, a wall display or speaker could be used as the output device. A microphone mounted in a room could be used as an input device, and additional memory may reside off the device. Any type of devices that can provide information to the physician and receive input from the physician can be used as an interactive device without departing from the scope of the invention as defined in the claims appended hereto.

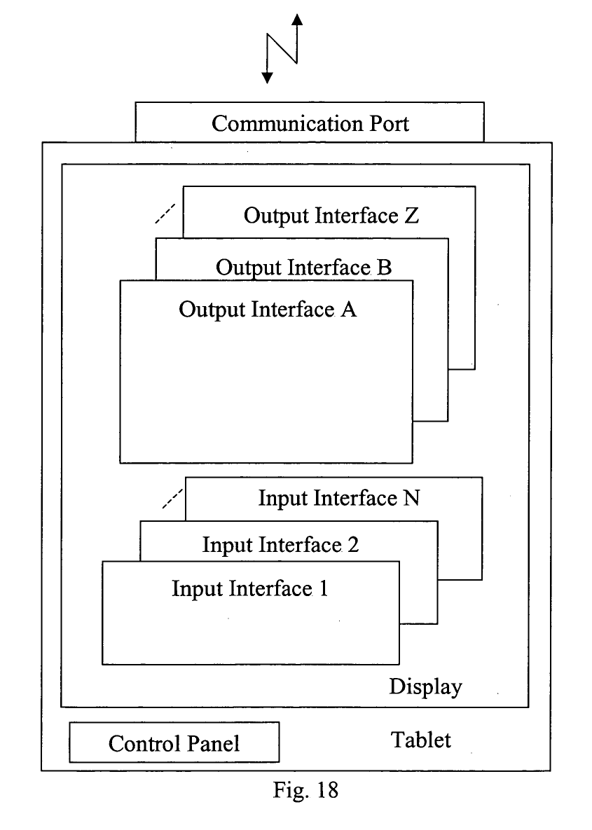

FIG. 18 shows a top view of the interactive device of FIG. 17. FIG. 18 shows a preferred interactive device in the form of a handheld computing device or tablet on which a physician interface is displayed. Tablet includes a touch sensitive screen for selecting items from a displayed screen, a pen stroke area (which may be the entire screen) for entering information using pens strokes, and a microphone for accepting speech commands or data from the physician. One or more connection ports allow direct connection of one or more devices such as an electronic thermometer or blood pressure measuring device.

A system for specifying medical diagnosis and treatment algorithms that may be integrated into a healthcare workflow, the system may include: (a) a coordination server having one or more rules for selecting at least one treatment algorithm based on medical and demographic information about a patient; and (b) an interface for providing a plurality of questions related to one or more medical findings, the questions may be asked of the patient or entered about the patient and potential orders that may be executed for the patient. The system may further enable access to a plurality of entities; each entity may specify one or more treatment algorithms to be included in one or more healthcare workflows.

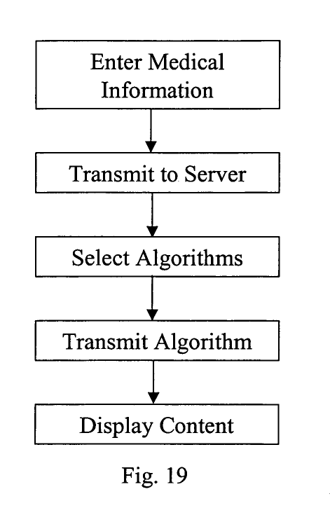

In an exemplary implementation, treatment algorithms are integrated into a healthcare workflow using the following steps, as shown in FIG. 19. In step 1 a user such as an HCP or patient accesses an interface such as an HCP interface or patient interface and enters medical findings. In step 2, entered findings and stored information about the patient are transmitted to a coordination server. In step 3, the coordination server selects at least one-treatment algorithm using rules for selecting at least one treatment algorithm based on medical and demographic information about a patient. In step 4, the coordination server transmits the at least one treatment algorithm to the interface. In step 5, the interface displays content specified by the at least one treatment algorithm.

A system for specifying medical diagnosis and treatment algorithms that may be integrated into a healthcare workflow, the system may include: (a) one or more coordination servers having one or more rules for selecting at least one treatment algorithm based on medical and demographic information about a patient; (b) an interface for providing a plurality of questions related to one or more medical findings, the questions may be asked of the patient or entered about the patient and potential orders that may be executed for the patient; and (c) a distribution server that distributes information from the interfaces to the one or more coordination servers, receives one or more treatment algorithms from the one or more coordination servers, and transmits these one or more treatment algorithms to the one or more interfaces to be included in one or more healthcare workflows. In an exemplary implementation, the one or more coordination servers are managed by or located at different organizations such as different DMAs.

In an exemplary implementation, treatment algorithms are integrated into a healthcare workflow using the following steps. In step 1 a user such as an HCP or patient accesses an interface such as an HCP interface or patient interface and enters medical findings. This accessing may also comprise inserting, swiping, and/or using a smart card. In step 2, the interface transmits entered findings and stored information about the patient to a distribution server. In step 3, a distribution server transmits entered findings and stored information about the patient to one or more coordination servers. In step 4, the one or more coordination servers select at least one-treatment algorithm using rules for selecting at least one treatment algorithm based on medical and demographic information about a patient. In step 5, the one or more coordination servers transmits the at least one treatment algorithm to the distribution server. In step 6, the distribution server transmits the at least one algorithm from the at least one servers to the interface. In step 7, the interface displays content specified by the at least one treatment algorithm.

One embodiment of the invention is based on a method for presenting disease management algorithms to at least one user, the method may comprise: receiving or storing disease management algorithms from at least two disease management advisors; selecting a subset of algorithms; and presenting the subset of algorithms to the at least one user. In the method step of selecting, the at least one user may input search with one or more selection criteria. Likewise, in the method step of presenting the subset of algorithms may be presented using a common display format. The method may further comprise an electronic healthcare workflow in which the step of selecting selects algorithms associated with medical or demographic information in an active patient's medical record. Alternatively, the method may further comprise an electronic healthcare workflow in which the step of selecting selects algorithms associated with one or more of the active patient's disease management advisors. In addition, the method may further comprise an electronic healthcare workflow in which the step of presenting comprises augmenting, modifying, or replacing the electronic healthcare workflow display with disease management algorithm information.

Another alternate embodiment of the invention is based on a computer-implemented method for integrating the display of disease management algorithms with an electronic medical record, the method comprising: providing an electronic healthcare workflow having one or more workflow tasks; providing one or more disease management algorithms; selecting one or more of the disease management algorithms; and presenting information or options specified by the disease management algorithms during the execution of one or more workflow tasks. The method step of selecting may further comprise utilizing relevant disease management algorithms based on the current patient's demographic information or medical information or both. The method step of presenting may further comprise augmenting, modifying, or replacing the options available or the data displayed at one or more workflow tasks in the electronic healthcare workflow. The displayed data may include a subset of algorithm elements that are displayed with the one or more workflow tasks are those algorithm elements, which are associated with the specific workflow task. The method step of presenting may further comprise using a plurality of actions, the actions include but not limited to displaying a banner, augmenting a data-input template, modifying a data-input template, replacing a data-input template, displaying an icon, displaying a warning, requiring acknowledgement of a warning, sending an alert message to the healthcare practitioner, providing an input device that may be activated to execute specified orders, providing an auditory notification, providing a tactile notification, changing the workflow order, and/or introducing one or more steps to the electronic healthcare workflow order.

Another embodiment of the invention provides a healthcare EMR system using an integrated workflow routine, a disease management engine, a plurality of disease management algorithms, and an interface to provide communication between one or more external disease management advisors and one or more healthcare providers/professionals/practitioners for servicing one or more patients. The communication may be provided through the interface in conjunction with the integrated workflow routine, which may utilize one or more workflow mechanisms.

For example, in a workflow mechanism, a disease management advisor creates an appointment within the healthcare EMR system for a patient to schedule a visit with a healthcare professional at a clinic for a healthcare professional-patient encounter. The disease management advisor transmits relevant information about the patient to the healthcare EMR system that is displayed by the integrated workflow routine during the patient's visit to the clinic, the disease management advisor transmits a disease management algorithm to the disease management engine for augmenting the integrated workflow routine for the healthcare EMR system, the disease management engine transmits refill requests for the patient to the integrated workflow routine, the disease management engine responsive to the disease management algorithm sends a message to the healthcare professional containing information or suggestions for improving his/her practice of medicine, for example, during a specific task from the disease management algorithm, the disease management engine sends a message to the healthcare professional containing information or suggestions for improving his/her decision for the specific task, the disease management advisor sends the healthcare professional a directive to send specific information about the patient to the disease management advisor upon completion of the healthcare professional-patient encounter, in response to the directive, the healthcare professional sends information about the patient to the disease management advisor, the integrated workflow routine sends information about the patient from the healthcare EMR system to the disease management engine, the information may include decisions or information entered into the integrated workflow routine during or for one or more past healthcare professional-patient encounters, and the disease management engine analyses these decisions to evaluate their effectiveness, to generate suggestions for improvement, or to provide data generated to a medical data gathering device controlled by the integrated workflow routine which sends data to the disease management advisor.

Another alternate embodiment of the invention is based on a healthcare EMR system, the healthcare EMR system may include an integrated workflow routine, a disease management engine, a plurality of disease management algorithms, and an interface to provide communication between one or more external disease management advisors, one or more healthcare providers/professionals/practitioners, and one or more patients. The communication may be provided through the interface in conjunction with the integrated workflow routine, which may utilize one or more workflow mechanisms.

For example, in a workflow mechanism, a disease management advisor directs a patient to make an appointment through the healthcare EMR system. Alternatively, a disease management advisor creates an appointment within the healthcare EMR system for a patient to schedule a visit with a healthcare professional at a clinic for a healthcare professional-patient encounter. The disease management advisor transmits relevant information about the patient to the healthcare EMR system that is displayed by the integrated workflow routine during the patient's visit to the clinic, the disease management advisor transmits a disease management algorithm to the disease management engine for augmenting the integrated workflow routine for the healthcare EMR system, the disease management engine transmits refill requests for the patient to the integrated workflow routine, the disease management engine responsive to the disease management algorithm sends a message to the healthcare professional containing information or suggestions for improving his/her practice of medicine, for example, during a specific task from the disease management algorithm, the disease management engine sends a message to the healthcare professional containing information or suggestions for improving his/her decision for the specific task, the disease management advisor sends the healthcare professional a directive to send specific information about the patient to the disease management advisor upon completion of the healthcare professional-patient encounter, in response to the directive, the healthcare professional sends information about the patient to the disease management advisor, the integrated workflow routine sends information about the patient from the healthcare EMR system to the disease management engine, the information may include decisions or information entered into the integrated workflow routine during or for one or more past healthcare professional-patient encounters, and the disease management engine analyses these decisions to evaluate their effectiveness, to generate suggestions for improvement, the disease management engine queries the patient to enter treatment compliance information and the health care system transmits the results to the disease management engine and/or to the healthcare professional for integration with the integrated workflow routine, in response, the disease management engine transmits instructions to the patient, to the healthcare professional, and/or to provide data generated to a medical data gathering device controlled by the integrated workflow routine which sends data to the disease management advisor.

Another embodiment of the invention is based on an electronic media, comprising a program for performing the methods of the invention. Another embodiment of the invention is based on a computer program, comprising computer or machine-readable program elements translatable for implementing the methods of the invention.

By providing an electronic tool into the hands of the physician during the patient encounter, the present invention allows real-time quality and efficiency guidance. Because the present invention assists rather than burdens the physician, he/she will use the system during the physician-patient encounter, so the diagnostic and treatment information are available electronically for automatic checking. Moreover, by providing the physician with authoritative guidelines for diagnoses and treatments, a standard level of care is provided. The physician is not constrained, however, to any diagnosis or treatment presented by the system. The physician is always free to enter the diagnosis and treatment elements that he deems appropriate.

Although the present invention and its advantages have been described in detail, it should be understood that the system and software represent the software system for healthcare professionals. For these reasons, various changes, substitutions and alterations may be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A system for specifying at least one medical diagnostic and treatment algorithm in a healthcare workflow, the at least one medical diagnostic and treatment algorithm being associated with at least one disease management entity, the system comprising:
at least one server having at least one medical diagnostic and treatment algorithm;
a user interface in communication with the at least one server, the user interface operable to display information associated with the healthcare workflow to a user; and
the at least one server operable to modify the healthcare workflow in accordance with the at least one medical diagnostic and treatment algorithm.

2. The system of claim 1 wherein the user is a healthcare professional.

3. The system of claim 1 wherein the user is a patient.

4. The system of claim 1 wherein the modification of the healthcare workflow is represented by the display of a banner.

5. The system of claim 1 wherein the modification of the healthcare workflow is represented by the display of a highlighted choice.

6. The system of claim 1 wherein the modification of the healthcare workflow is represented by the display of a question.

7. The system of claim 1 wherein the modification of the healthcare workflow is represented by the display of a notification of a drug trial.

8. The system of claim 1 wherein the modification of the healthcare workflow is represented by the display of a recommended step therapy.

9. The system of claim 1 wherein the user interface is a portable device.

10. A portable device for integrated disease management, the portable device being in communication with at least one server, the at least one server having at least one medical diagnostic and treatment algorithm associated with a healthcare workflow, the at least one medical diagnostic and treatment algorithm being associated with at least one disease management entity, the portable device comprising:
a display, the display displaying at least one interface associated with the healthcare workflow to a user; and
a communications circuitry for communicating with the at least one server, the communications circuitry sending information associated with the healthcare workflow to the at least one server, the communications circuitry receiving an information from the at least one server, the information being associated with a modification in the healthcare workflow, the display modifying the at least one interface to represent the modification in the healthcare workflow.

11. The portable device of claim 10 wherein the user is a healthcare professional.

12. The portable device of claim 10 wherein the user is a patient.

13. The portable device of claim 10 wherein the modification of the at least one interface is represented by a banner.

14. The portable device of claim 10 wherein the modification of the at least one interface is represented by the display of a highlighted choice.

15. The portable device of claim 10 wherein the modification of the at least one interface is represented by the display of a new question.

16. The portable device of claim 10 wherein the modification of the at least one interface is represented by the display of a notification of a drug trial.

17. The portable device of claim 10 wherein the modification of the at least one interface is represented by the display of a recommended step therapy.

18. A method for selectively modifying a healthcare workflow, the method comprising:
collecting medical information from a user with an interactive device;
transmitting the medical information to a server from the interactive device;
selecting a medical diagnostic and treatment algorithm for a set of at least one medical diagnostic and treatment algorithms;
transmitting medical diagnostic and treatment information associated with the medical diagnostic and treatment algorithm to the interactive device; and
selectively displaying the medical diagnostic and treatment information to the user with the interactive device.

19. The method of claim 18 wherein the user is a healthcare professional.

20. The method of claim 18 wherein the user is a patient.

21. The method of claim 18 wherein the medical diagnostic and treatment information is represented by a banner.

22. The method of claim 18 wherein the medical diagnostic and treatment information is represented by the display of a highlighted choice.

23. The method of claim 18 wherein the medical diagnostic and treatment information is represented by the display of a new question.

24. The method of claim 18 wherein the medical diagnostic and treatment information is represented by the display of a notification of a drug trial.

25. The method of claim 18 wherein the medical diagnostic and treatment information is represented by the display of a recommended step therapy.

26. The method of claim 18 wherein the step of collecting medical information comprises interfacing with a smart card.

27. A program storage device readable by a machine, tangibly embodying a program of instructions executable by the machine to perform methods steps for selectively modifying a healthcare workflow, said method steps comprising:
collecting medical information from a user with an interactive device;
transmitting the medical information to a server from the interactive device;
selecting a medical diagnostic and treatment algorithm for a set of at least one medical diagnostic and treatment algorithms;
transmitting medical diagnostic and treatment information associated with the medical diagnostic and treatment algorithm to the interactive device; and
selectively displaying the medical diagnostic and treatment information to the user with the interactive device.
